(19) United States
(12) Patent Application Publication
(10) Pub. No.: US 2016/0089117 A1
(43) Pub. Date: Mar. 31, 2016

(54) ULTRASOUND IMAGING APPARATUS AND METHOD USING SYNTHETIC APERTURE FOCUSING

(71) Applicant: SAMSUNG ELECTRONICS CO., LTD., Suwon-si (KR)

(72) Inventor: Kang-sik KIM, Seongnam-si (KR)

(73) Assignee: SAMSUNG ELECTRONICS CO., LTD., Suwon-si (KR)

(21) Appl. No.: 14/612,721

(22) Filed: Feb. 3, 2015

(30) Foreign Application Priority Data
Sep. 25, 2014 (KR) 10-2014-0128280

(51) Int. Cl. A61B 8/00; A61B 8/08; A61B 8/14

(52) U.S. Cl. CPC A61B 8/54; A61B 8/14; A61B 8/5207

(57) ABSTRACT

The ultrasound diagnostic apparatus includes: an ultrasound transceiver supplying a driving signal to at least one transducer included in a probe and acquiring ultrasound data corresponding to at least one ultrasound echo signal received by the transducer; a controller performing control to acquire the ultrasound data based on at least one first ultrasound echo signal acquired by the ultrasound transceiver in response to an ultrasound signal transmitted to an object by the probe at a first position and at least one second ultrasound echo signal acquired by the ultrasound transceiver in response to an ultrasound signal transmitted to the object by the probe at a second position that is different from the first position; and an image processor generating an ultrasound image by using the ultrasound data.

### CROSS-REFERENCE TO RELATED APPLICATION

This application claims priority from Korean Patent Application No. 10-2014-0128280, filed on Sep. 25, 2014, in the Korean Intellectual Property Office, the disclosure of which is incorporated herein in its entirety by reference.

### BACKGROUND

1. Field

One or more exemplary embodiments relate to ultrasound imaging apparatuses and methods using synthetic aperture focusing, and more particularly, to apparatuses and methods for generating an ultrasound image of an object.

2. Description of the Related Art

An ultrasound diagnostic apparatus transmits ultrasound signals generated by transducers of a probe to an object and receives echo signals reflected from the object, thereby obtaining images of the object (e.g., tomography of soft tissues or blood flow). In particular, an ultrasound diagnostic apparatus may be used for medical purposes including observation of the interval areas of an object, detection of foreign substances, and diagnosis. Such an ultrasound diagnostic apparatus may display information regarding an object in real-time. Furthermore, unlike the use of X-rays, an ultrasound diagnostic apparatus does not involve any radioactive exposure, thus being safe. Therefore, an ultrasound diagnostic apparatus is widely used together with other types of imaging diagnostic apparatuses, including a computed tomography (CT) scanner, a magnetic resonance imaging (MRI) apparatus, a nuclear medicine apparatus, etc.

The quality of an ultrasound image may be determined by resolution, a contrast, penetration, a frame rate, and the like, and the performance thereof may vary according to transmission/reception methods, image forming methods, signal processing methods, and characteristics of circuits. Among these factors, resolution is an important index for determining the quality of an ultrasound image. Therefore, methods and apparatuses are needed for improving the resolution of an ultrasound image.

### SUMMARY

One or more exemplary embodiments include ultrasound diagnostic apparatuses and methods for acquiring an ultrasound image with increased resolution by using a probe having a limited aperture size.

According to one or more exemplary embodiments, ultrasound image data may be acquired by transmitting and receiving ultrasound signals at different positions by moving a probe with a limited aperture size. Thus, the probe with an aperture size increased by the movement distance of the probe may provide the same effect as a probe with a larger aperture size in acquiring an ultrasound image.

One or more exemplary embodiments include an ultrasound image displaying method for obtaining a high-resolution ultrasound image by using this effect and an ultrasound diagnostic apparatus using the ultrasound image displaying method.

One or more exemplary embodiments include an ultrasound image displaying method for obtaining a high-resolution ultrasound image by transmitting ultrasound signals at different positions by moving a probe to increase a virtual size of the probe by the movement distance of the probe and an ultrasound diagnostic apparatus using the ultrasound image displaying method.

According to one or more exemplary embodiments, an ultrasound diagnostic apparatus includes: an ultrasound transceiver supplying a driving signal to at least one transducer included in a probe and acquiring at least one piece of ultrasound data corresponding to at least one ultrasound echo signal received by the at least one transducer; a controller performing control to acquire the at least one piece of ultrasound data based on at least one first ultrasound echo signal acquired by the ultrasound transceiver in response to an ultrasound signal transmitted to an object by the probe at a first position and at least one second ultrasound echo signal acquired by the ultrasound transceiver in response to an ultrasound signal transmitted to the object by the probe at a second position that is different from the first position; and an image processor generating an ultrasound image by using the ultrasound data.

The controller may perform control to acquire the at least one piece of ultrasound data by synthetic aperture focusing of the at least one first ultrasound echo signal and the at least one second ultrasound echo signal.

The controller may perform control such that the probe is formed to have a synthetic aperture corresponding to the sum of an aperture of the probe at the first position and an aperture of the probe at the second position.

When one of the at least one first ultrasound echo signal and one of the at least one second ultrasound echo signal is determined to be the same as each other, the image processor may delete one of information about the one of the at least of first ultrasound echo signal and information about the one of the at least of second ultrasound echo signal.

The image processor may apply a predetermined weight to the at least one ultrasound echo signal, which is determined to be the same, among at least one of the at least one first ultrasound echo signal and at least one of the at least one second ultrasound echo signal.

The image processor may generate the ultrasound data by applying different weights to the at least one first ultrasound echo signal and the at least one second ultrasound echo signal.

The image processor may generate a panoramic image including the ultrasound data generated based on the at least one first ultrasound echo signal and the at least one second ultrasound echo signal.

According to one or more exemplary embodiments, an ultrasound diagnostic method includes: transmitting and receiving at least one ultrasound signal to and from an object by a probe at a first position; transmitting and receiving at least one ultrasound signal to and from the object by the probe at a second position that is different from the first position; and acquiring at least one piece of ultrasound data based on at least one first ultrasound echo signal received at the first position and at least one second ultrasound echo signal received at the second position.

The acquiring of the at least one piece of ultrasound data may include acquiring the at least one piece of ultrasound data by synthetic aperture focusing of the at least one first ultrasound echo signal and the at least one second ultrasound echo signal.

The acquiring of the at least one piece of ultrasound data may include performing a calculation to acquire ultrasound data by using the probe formed to have a synthetic aperture corresponding to the synthesis of an aperture of the probe at the first position and an aperture of the probe at the second position.

The acquiring of the at least one piece of ultrasound data may include deleting one of information about the first ultrasound echo signal and information about the second ultrasound echo signal when one of the at least one first ultrasound echo signal and one of the at least one second ultrasound echo signal is determined to be the same as each other.

The acquiring of the at least one piece of ultrasound data may include applying a predetermined weight to the ultrasound echo signal, which is determined to be the same, among at least one of the at least one first ultrasound echo signal and at least one of the at least one second ultrasound echo signal.

The acquiring of the at least one piece of ultrasound data may include acquiring the ultrasound data by applying different weights to the at least one first ultrasound echo signal and the at least one second ultrasound echo signal.

The acquiring of the at least one piece of ultrasound data may include generating a panoramic image including the ultrasound data generated based on the at least one first ultrasound echo signal and the at least one second ultrasound echo signal.

According to one or more exemplary embodiments, a non-transitory computer-readable recording medium stores a program that performs the above ultrasound diagnostic method when executed by a computer.

### BRIEF DESCRIPTION OF THE DRAWINGS

The above and/or other aspects will become more apparent by describing certain exemplary embodiments, with reference to the accompanying drawings, in which:

FIG. 1 is a block diagram illustrating a configuration of an ultrasound diagnostic apparatus according to an exemplary embodiment;

FIG. 2 illustrates an exemplary embodiment of dynamic transmit focusing;

FIG. 3 is a block diagram illustrating components included in an ultrasound diagnostic apparatus according to an exemplary embodiment;

FIGS. 4A and 4B illustrate an exemplary embodiment of synthetic aperture focusing;

FIG. 5 illustrates acquiring ultrasound data by synthetic aperture focusing of ultrasound signals transmitted and received by using a probe located at different positions, according to an exemplary embodiment;

FIG. 6 illustrates an ultrasound echo signal transmitted and received at a first position or a second position, which is determined to be the same, according to an exemplary embodiment;

FIG. 7 illustrates generating a panoramic image by using ultrasound data generated at different positions via the movement of a probe, according to an exemplary embodiment;

FIG. 8 is a flowchart of an ultrasound data acquiring method using synthetic aperture focusing to acquire ultrasound data by transmitting and receiving ultrasound signals at different positions via the movement of a probe, according to an exemplary embodiment;

FIG. 9 is a flowchart of a method of acquiring ultrasound data based on information about an ultrasound echo signal other than an ultrasound echo signal, which is determined to be the same, among at least one first ultrasound echo signal acquired in response to an ultrasound signal transmitted to an object at a first position and at least one second ultrasound echo signal acquired in response to an ultrasound signal transmitted to the object at a second position, according to an exemplary embodiment;

FIG. 10 is a flowchart of a method of acquiring ultrasound data by applying a predetermined weight to information about a first ultrasound echo signal and a second ultrasound echo signal determined to be the same, according to an exemplary embodiment;

FIG. 11 is a flowchart of a method of acquiring ultrasound data by applying different weights to a first ultrasound echo signal and a second ultrasound echo signal, according to an exemplary embodiment; and

FIG. 12 is a flowchart of a method of generating a panoramic image by using ultrasound data acquired by synthetic aperture focusing of ultrasound signals transmitted and received by using a probe located at different positions, according to an exemplary embodiment.

### DETAILED DESCRIPTION

Certain exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

As used herein, expressions such as “at least one of,” when preceding a list of elements, modify the entire list of elements and do not modify the individual elements of the list.

When something “comprises” or “includes” a component, another component may be further included unless specified otherwise. Also, terms such as “ . . . unit”, “ . . . module”, or the like refer to units that perform at least one function or operation, and the units may be implemented as hardware or software or as a combination of hardware and software.

Throughout the specification, an “ultrasound image” refers to an image of an object, which is acquired by using ultrasonic waves. Also, an “object” may include a person or an animal, or a part of a person or an animal. For example, the object may include organs such as a liver, a heart, a womb, a brain, a breast, an abdomen, or the like, or a blood vessel. Also, the object may include a phantom. The phantom may refer to a material having a volume that is approximately the intensity and effective atomic number of a living thing, and may include a spherical phantom having a property similar to a human body.

Also, a “user” may be, but is not limited to, a medical expert including a doctor, a nurse, a medical laboratory technologist, a medial image expert, and a technician who repairs a medical apparatus.

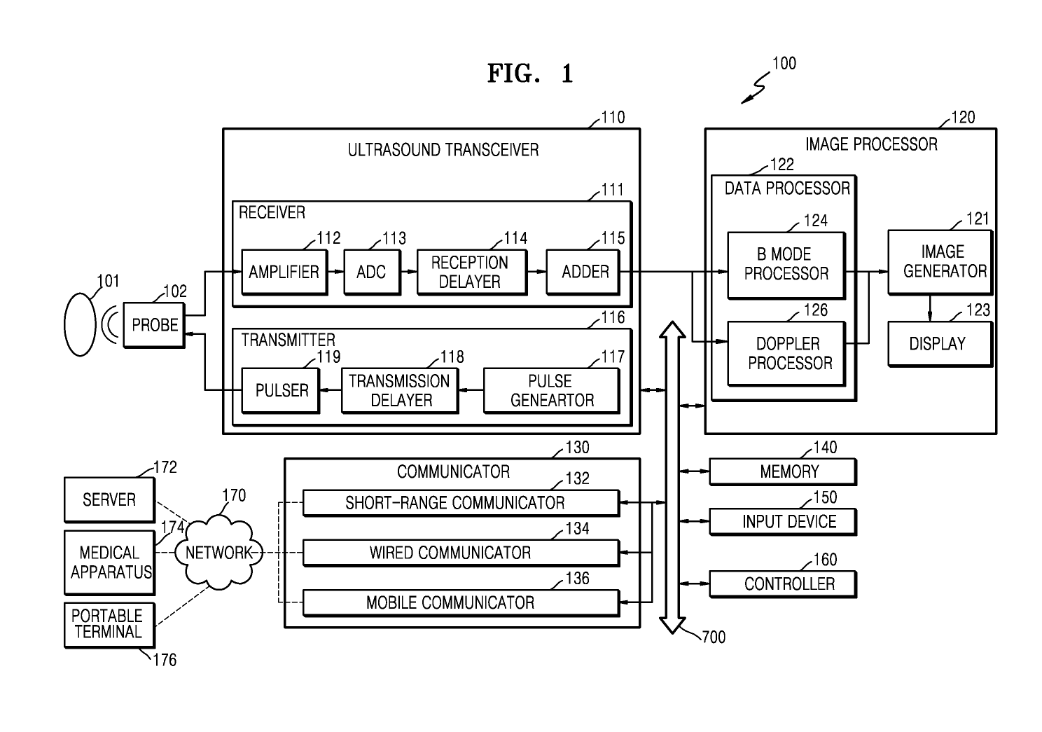

FIG. 1 is a block diagram illustrating a configuration of an ultrasound diagnostic apparatus 100 according to an exemplary embodiment. The ultrasound diagnostic apparatus 100 according to an exemplary embodiment may include a probe or probes 102, an ultrasound transceiver 110, an image processor 120, a communicator 130, a memory 140, an input device 150, and a controller 160, where the components stated above may be connected to one another via buses 700.

The ultrasound diagnostic apparatus 100 may be embodied a cart type apparatus and/or a portable apparatus. Examples of portable ultrasound diagnostic apparatuses may include a picture archiving and communication system (PACS) viewer, a smart phone, a laptop computer, a personal digital assistant (PDA), and a tablet personal computer (PC). However, exemplary embodiments are not limited thereto.

The probe 102 transmits an ultrasound signal to an object 101 according to a driving signal applied from the ultrasound transceiver 110 and receives an echo signal reflected from the object 101. The probe 102 includes a plurality of transducers, and the plurality of transducers oscillates according to an electrical signal transmitted thereto and generates an ultrasound wave, that is, acoustic energy. A scan line is defined by each of the plurality of transducers. For example, a plurality of scan lines of linearly arranged transducers may be defined as a plurality of scan lines that are perpendicular to the arrangement direction of transducers. This example is described based on a two-dimensional (2D) ultrasound image in terms of a lateral direction (x axis), that is, the arrangement direction of transducers and an axial direction (z axis) constituted by scan lines, and according to various embodiments, the scan lines may be divided into a y axis in addition to the x axis and the z axis, for generation of a three-dimensional (3D) ultrasound image. The probe 102 may be connected to the main body of the ultrasound diagnostic apparatus 100 by wire or wirelessly. According to exemplary embodiments, the ultrasound diagnostic apparatus 100 may include a plurality of probes 102.

A transmitter 116 supplies a driving signal to the probe 102 and includes a pulse generator 117, a transmission delayer 118, and a pulser 119. The pulse generator 117 generates pulses for forming transmission ultrasound waves according to a predetermined pulse repetition frequency (PRF), and the transmission delayer 118 applies a delay time for determining transmission directionality to the pulses. The pulses to which the delay time is applied correspond to a plurality of piezoelectric vibrators included in the probe 102, respectively. The pulser 119 applies a driving signal (or a driving pulse) to the probe 102 at a timing corresponding to each pulse to which the delay time is applied.

A reception unit 111 generates ultrasound data by processing echo signals received from the probe 102 and may include an amplifier 112, an analog-digital converter (ADC) 113, a reception delayer 114, and a adder 115. The amplifier 112 amplifies echo signals in each channel, and the ADC 113 analog-digital converts the amplified echo signals. The reception delayer 114 applies delay times for determining reception directionality to the digital-converted echo signals, and the adder 115 generates ultrasound data by summing the echo signals processed by the reception delayer 114. Also, according to exemplary embodiments, the reception unit 111 may omit the amplifier 112. In other words, when the sensitivity of the probe 102 or the capability to process bits by the ADC 113 is enhanced, the amplifier 112 may be omitted.

The image processor 120 generates an ultrasound image by scan-converting ultrasound data generated by the ultrasound transceiver 110 and displays the ultrasound image. The ultrasound image may include not only a gray-scale image obtained by scanning the object 101 in an amplitude (A) mode, a brightness (B) mode, and a motion (M) mode, but also a Doppler image representing a motion of the object 101 by using a Doppler effect. The Doppler image may include a bloodstream Doppler image (also referred to as a color Doppler image) representing a flow of blood, a tissue Doppler image representing a motion of a tissue, and a spectral Doppler image representing a movement speed of the object 101 in a waveform.

A B mode processor 124 extracts B mode components from ultrasound data and processes the B mode components. An image generator 121 may generate an ultrasound image representing signal intensities as brightness based on the B mode components extracted by the B mode processor 124.

Likewise, a Doppler processor 126 may extract Doppler components from ultrasound data, and the image generator 121 may generate a Doppler image representing a motion of the object 101 as colors or waveforms based on the extracted Doppler components.

The image generator 121 according to an exemplary embodiment may generate a 3D ultrasound image through volume-rendering of volume data and may also generate an elasticity image that visualizes the deformation of the object 101 due to a pressure. In addition, the image generator 121 may display various additional information in or on an ultrasound image by using texts and graphics. The generated ultrasound image may be stored in the memory 140.

A display 123 displays the generated ultrasound image. The display 123 may display an ultrasound image, and/or various information processed by the ultrasound diagnostic apparatus 100 on a screen via a graphic user interface (GUI). The ultrasound diagnostic apparatus 100 may include two or more displays 123 according to exemplary embodiments.

The communicator 130 is connected by wire or wirelessly to a network 170 to communicate with an external device or a server. The communicator 130 may exchange data with a hospital server or other medical apparatuses in a hospital connected through a PACS. The communicator 130 may perform data communication according to the Digital Imaging and Communications in Medicine (DICOM) standard.

The communicator 130 may transmit and receive data related to diagnosis of the object 101, e.g., an ultrasound image, ultrasound data, and Doppler data of the object 101, via the network 170 and may also transmit and receive medical images obtained by other medical apparatuses, e.g., a computed tomography (CT) image, a magnetic resonance imaging (MRI) image, and an X-ray image. In addition, the communicator 130 may receive information related to diagnosis history or a treatment schedule of a patient from a server and use the information to diagnose the object 101. In addition, the communicator 130 may perform data communication not only with a server or a medical apparatus in a hospital, but also with a portable terminal of a doctor or a patient.

The communicator 130 may be connected by wire or wirelessly to the network 170 to exchange data with a server 172, a medical apparatus 174, or a portable terminal 176. The communicator 130 may include one or more components that enable communication with external devices, and may include, for example, a short-range communicator 132, a wired communicator 134, and a mobile communicator 136.

The short-range communicator 132 refers to a module for short-range communication within a predetermined distance. Examples of short-range communication techniques according to an exemplary embodiment may include wireless LAN, Wi-Fi, Bluetooth, Zigbee, Wi-Fi Direct (WFD), ultra wideband (UWB), infrared data association (IrDA), Bluetooth Low Energy (BLE), and near field communication (NFC); however, exemplary embodiments are not limited thereto.

The wired communicator 134 refers to a module for communication using electrical signals or optical signals. Examples of wired communication techniques according to an exemplary embodiment may include a pair cable, a coaxial cable, an optical fiber cable, and an Ethernet cable.

The mobile communicator 136 transmits and receives wireless signals to and from at least one of a base station, an external terminal, and a server on a mobile communication network. Herein, the wireless signals may include voice call signals, video call signals, or various types of data for transmission and reception of text/multimedia messages.

The memory 140 stores various data processed by the ultrasound diagnostic apparatus 100. For example, the memory 140 may store medical data related to diagnosis of the object 101, such as ultrasound data and ultrasound images that are input or output and may also store algorithms or programs to be executed in the ultrasound diagnostic apparatus 100.

The memory 140 may be embodied as any of various storage media such as a flash memory, a hard disk drive, and an electrically erasable programmable read-only memory (EEPROM). The ultrasound diagnostic apparatus 100 may utilize web storage or a cloud server that functions as the memory 140 online.

The input device 150 refers to a means via which a user inputs data for controlling the ultrasound diagnostic apparatus 100. The input device 150 may include hardware components, such as a keypad, a mouse, a touch panel, a touch screen, a track ball, and a jog switch. However, exemplary embodiments are not limited thereto, and the input device 150 may further include various other input means, such as an electrocardiogram measuring module, a respiration measuring module, a voice recognition sensor, a gesture recognition sensor, a fingerprint recognition sensor, an iris recognition sensor, a depth sensor, and a distance sensor.

The controller 160 may control overall operations of the ultrasonic diagnostic apparatus 100. In other words, the controller 160 may control operations among the probe 102, the ultrasound transceiver 110, the image processor 120, the communicator 130, the memory 140, and the input device 150 illustrated in FIG. 1.

All or some of the probe 102, the ultrasound transceiver 110, the image processor 120, the communicator 130, the memory 140, the input device 150, and the controller 160 may be operated by software modules. However, exemplary embodiments are not limited thereto, and some of the above components may be operated by hardware modules. Also, at least one of the ultrasound transceiver 110, the image processor 120, and the communicator 130 may be included in the controller 160; however, exemplary embodiments are not limited thereto.

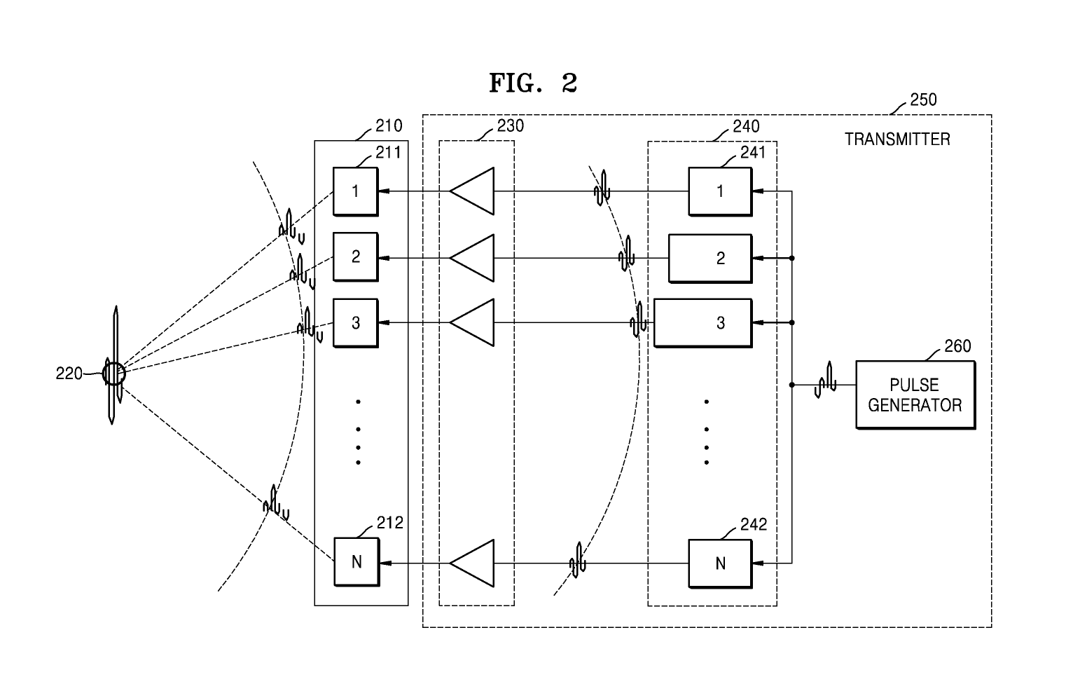

FIG. 2 is a diagram illustrating a transmitter 250 according to an exemplary embodiment. Since a probe 210 and the transmitter 250 illustrated in FIG. 2 correspond respectively to the probe 102 and the transmitter 116 illustrated in FIG. 1, redundant descriptions thereof will be omitted herein.

Referring to FIG. 2, in order to focus ultrasound signals, which are transmitted from first to Nth transducers 211 to 212 included in the probe 210, on a first focus region 220, the transmitter 250 may provide transmission signals, to which different transmission delay times are applied, to the first to Nth transducers 211 to 212 respectively. Herein, the first to Nth transducers 211 to 212 have the shape of a transducer array that is arranged along one axis. In detail, a pulse generator 260 generates transmission signals, and the generated transmission signals are transmitted to first to Nth delay elements 241 to 242 included in a transmission delayer 240. Different transmission delay times may be applied to first to Nth transmission signals transmitted to the first to Nth delay elements 241 to 242. Outputs of the first to Nth delay elements 241 to 242, to which different transmission delay times are applied, may be transmitted through a pulser 230 to the first to Nth transducers 211 to 212. Thus, ultrasound signals output from the first to Nth transducers 211 to 212 may be controlled to have the same phase based on the different transmission delay times when reaching the first focus region 220 in the object 101, and the focusing of the ultrasound signals on the first focus region 220 may be defined as transmit focusing. However, the configurations of FIG. 2 for transmit focusing are merely an exemplary embodiment, and various modifications by those of ordinary skill in the art may also be included in the scope of exemplary embodiments.

The transmitter 250 may correspond to the transmitter 116 illustrated in FIG. 1 or may be separate therefrom. The probe 210 may correspond to the probe 102 illustrated in FIG. 1 or may be separate therefrom. The pulse generator 260 may correspond to the pulse generator 117 illustrated in FIG. 1 or may be separate therefrom. The transmission delayer 240 may correspond to the transmission delayer 118 illustrated in FIG. 1 or may be separate therefrom. The pulser 230 may correspond to the pulser 119 illustrated in FIG. 1 or may be separate therefrom.

A related art dynamic focusing (CDF) technique, which constructs one scan line by performing one transmission/reception process, may obtain a focused signal in all regions on a scan line with respect to a received beam, but resolution is degraded in other regions because a fixed focusing point is provided in transmission. On the other hand, a synthetic aperture (SA) technique may obtain a higher-resolution image than the CDF technique because it may obtain a focused signal in all regions of transmission and reception.

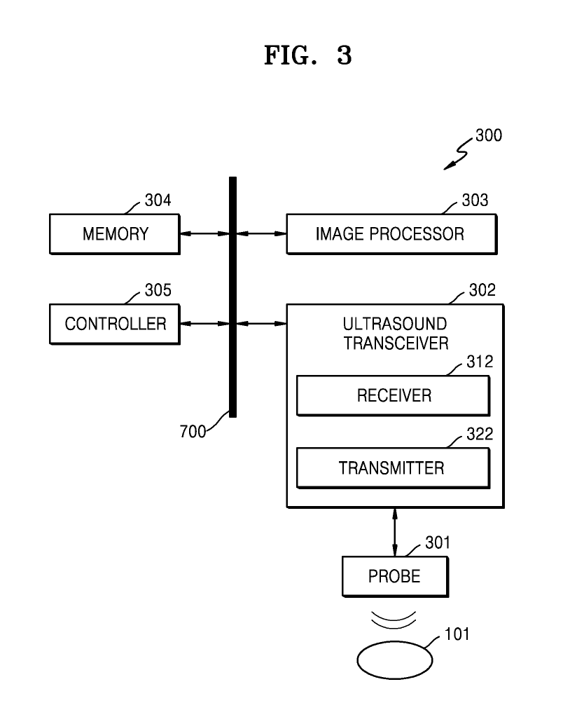

Synthetic aperture focusing is a method of constructing each scan line by transmitting and receiving through transducers and applying proper delay times to received signals prior to combination of the received signals. Due to the characteristics of synthetic aperture focusing, an improvement in side resolution of an ultrasound image by synthetic aperture focusing is influenced by the size of a probe. However, the expansion of a probe size for resolution improvement may be physically limited, and an increased probe size may cause inconvenience in use. FIG. 3 illustrates an ultrasound diagnostic apparatus for acquiring an ultrasound image with improved resolution by using a probe that has a limited size when using synthetic aperture focusing.

FIG. 3 is a block diagram illustrating components included in an ultrasound diagnostic apparatus according to an exemplary embodiment.

Referring to FIG. 3, an ultrasound diagnostic apparatus 300 according to an exemplary embodiment includes an ultrasound transceiver 302, a controller 305, and an image processor 303. The ultrasound diagnostic apparatus 300 may correspond to the ultrasound diagnostic apparatus 100 illustrated in FIG. 1. In detail, the ultrasound transceiver 302, the controller 305, and the image processor 303 may correspond respectively to the ultrasound transceiver 110, the controller 160, and the image processor 120 illustrated in FIG. 1. Thus, redundant descriptions of the same features as in FIG. 1 will be omitted in describing the ultrasound diagnostic apparatus 300.

The ultrasound transceiver 302 supplies a driving signal to at least one transducer included in a probe 301 and acquires at least one piece of ultrasound data corresponding to at least one ultrasound echo signal received by the at least one transducer.

In the ultrasound diagnostic apparatus 300, the controller 305 performs control to acquire the at least one piece of ultrasound data based on at least one first ultrasound echo signal acquired by the ultrasound transceiver 302 in response to an ultrasound signal transmitted to an object 101 by the probe 301 at a first position and at least one second ultrasound echo signal acquired by the ultrasound transceiver 302 in response to an ultrasound signal transmitted to the object 101 by the probe 301 at a second position that is different from the first position.

The image processor 303 generates an ultrasound image by using the ultrasound data.

The ultrasound diagnostic apparatus 300 may include at least one of the probe 301 and a memory 304.

The probe 301 transmits an ultrasound signal to the object 101 and receives an ultrasound echo signal reflected from the object 101. According to an exemplary embodiment, the probe 301 may transmit and receive an ultrasound signal at the first position and then transmit and receive an ultrasound signal at the second position that is different from the first position. An array of transducers in the probe 301 may be a linear array, a curvilinear array, or a convex array but is not limited thereto. The probe 301 may correspond to the probe 102 or may be separate therefrom. The probe 301 may be formed as a separate device without being included in the ultrasound diagnostic apparatus 300.

The ultrasound transceiver 302 may generate a transmission signal for triggering an ultrasound signal to be transmitted from the probe 301 to the object 101 and may generate ultrasound data by processing an ultrasound echo signal received by the probe 301. Focusing may be performed to generate the ultrasound data. That is, a transmitter 322 included in the ultrasound transceiver 302 may perform transmit-focusing of an ultrasound signal, which is transmitted by the probe 301, on the object 101 by applying a proper time delay to a transmission signal, and a reception unit 312 included in the ultrasound transceiver 302 may perform receive-focusing of a received ultrasound signal on a certain point by applying a proper time delay in generating ultrasound data based on a received ultrasound echo signal. According to an exemplary embodiment, the ultrasound echo signal on which the ultrasound data is based may include an ultrasound echo signal received by the probe 301 at the first position and an ultrasound echo signal received by the probe 301 at the second position that is different from the first position, and a time delay may be applied to perform receive-focusing of the received ultrasound echo signal on a certain point of the object 101. The ultrasound data may be generated based on the ultrasound echo signal with respect to a scan line defined from the transducer in the probe 301. The ultrasound transceiver 302 may correspond to the ultrasound transceiver 110 illustrated in FIG. 1 or may be separate therefrom.

The image processor 303 generates an ultrasound image based on the ultrasound data generated by the ultrasound transceiver 302. The ultrasound image may be generated based on the ultrasound data generated by the ultrasound transceiver 302 with respect to a scan line defined from a reception transducer in the probe 301, and the scan line may be a reference line of the axial direction that is perpendicular to a reference line of the arrangement direction of transducers. According to an exemplary embodiment, the ultrasound transceiver 302 may generate ultrasound data based on an ultrasound echo signal reflected from the object 101 in response to an ultrasound signal transmitted by each transmission transducer of the probe 301 at the first position, and may generate ultrasound data based on an ultrasound echo signal reflected from the object 101 in response to an ultrasound signal transmitted by each transmission transducer of the probe 301 at the second position that is different from the first position. A plurality of generated ultrasound echo signals corresponding to the first position and the second position are stored in the memory 304 and used by the image processor 303 to generate a high-resolution ultrasound image. That is, the image processor 303 acquires ultrasound data by synthetic-focusing of ultrasound echo signals acquired via the movement of the probe 301. Since the size of an aperture is expanded by the movement distance of the probe 301 and the resolution of an ultrasound image is proportional to the size of an aperture, the image processor 303 may increase the resolution of an acquired ultrasound image by acquiring an ultrasound image by using ultrasound data that is synthetic-focused via the movement of the probe 301.

The memory 304 may store information including the ultrasound data corresponding to the ultrasound echo signal. According to an exemplary embodiment, the ultrasound data stored in the memory 304 may be used in a time delay process by the reception unit 312, and the ultrasound data time-delayed and stored in the memory 304 may be used by the image processor 303 to generate an ultrasound image.

The controller 305 may control the ultrasound transceiver 302 and the image processor 303 to generate an ultrasound image. According to an exemplary embodiment, the controller 305 controls the ultrasound transceiver 302 to perform focusing on a certain point of the object 101 by performing transmit-focusing or receive-focusing in the ultrasound transceiver 302 when the probe 301 transmits and receives ultrasound signals at the first position and the second position, and controls the image processor 303 to generate an ultrasound image by using the ultrasound data generated by the ultrasound transceiver 302.

Synthetic aperture focusing may be used to obtain a high-resolution ultrasound image. By using synthetic aperture focusing, high lateral resolution may be provided and the effective depth of an image may be increased. In addition, synthetic aperture focusing may be usefully used to improve the lateral resolution of an image.

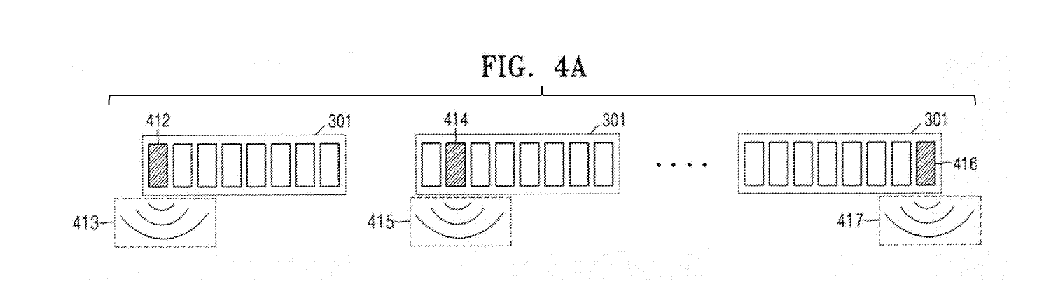

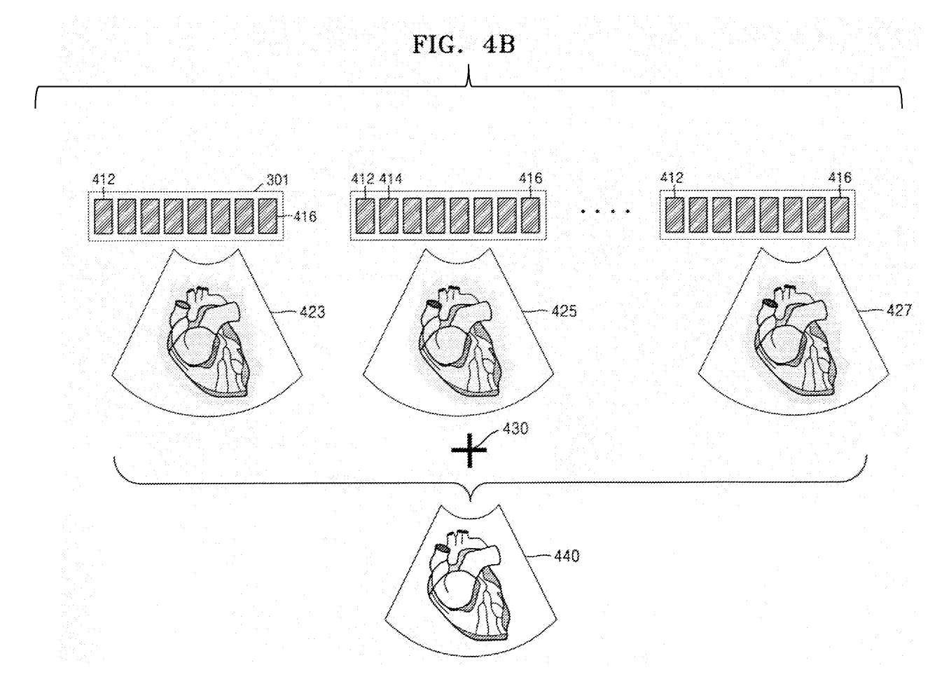

Hereinafter, synthetic aperture focusing used in the ultrasound diagnostic apparatus 300 according to an exemplary embodiment will be described in detail with reference to FIGS. 4A and 4B.

FIGS. 4A and 4B illustrate an exemplary embodiment of synthetic aperture focusing according to an exemplary embodiment.

Synthetic aperture focusing is an ultrasound imaging method that generates a final ultrasound image 440 by acquiring a plurality of ultrasound images 423, 425, and 427 based on ultrasound echo signals received respectively from a plurality of transducers 412, 414, and 416 included in the probe 301 and synthesizing the plurality of ultrasound images 423, 425, and 427.

Referring to FIGS. 4A and 4B, in order to perform synthetic aperture focusing, the ultrasound transceiver 302 does not need to transmit ultrasound signals by simultaneously focusing the ultrasound signals on one focus at a plurality of transducers as illustrated in FIG. 2, and may transmit ultrasound signals not simultaneously but sequentially at the transducers in the probe 301 as illustrated in FIG. 3. In detail, the transducers may transmit ultrasound signals sequentially, for example, the second transducer 414 transmits a second ultrasound signal 415 after the first transducer 412 transmits a first ultrasound signal 413. In detail, when the probe 301 includes N transducers, the first to Nth transducers 412 to 416 may transmit first to Nth ultrasound signals 413 to 417 respectively and sequentially. When each transducer transmits an ultrasound signal, an ultrasound echo signal, which is reflected from the object 101 whenever one ultrasound signal is transmitted, may be received by all of the transducers of the probe 301. That is, when the probe 301 includes N transducers, N ultrasound echo signals related to N ultrasound signals transmitted respectively by the N transducers (from a first ultrasound echo signal related to the first ultrasound signal 413 transmitted by the first transducer 412 to an Nth ultrasound echo signal related to the Nth ultrasound signal 417 transmitted by the Nth transducer 416) are received by all of the N transducers. Ultrasound images 423, 425, and 427 may be generated based on ultrasound data that is acquired based on the ultrasound echo signals related to the transmitted ultrasound signals, respectively. However, the ultrasound image generated based on each ultrasound echo signal has low resolution because it is generated by receive-focusing an ultrasound echo signal corresponding to an ultrasound signal transmitted by one transducer. Hereinafter, an image acquired by using an ultrasound echo signal corresponding to an ultrasound signal transmitted by one transducer will be referred to as a low-resolution image. A high-resolution ultrasound image 440 may be generated by combining or synthesizing (operation 430) a plurality of low-resolution images acquired by ultrasound-scanning the same object 101 by using various methods. The synthetic aperture focusing is merely an example, and exemplary embodiments are not limited to the synthetic aperture focusing illustrated in FIGS. 4A and 4B.

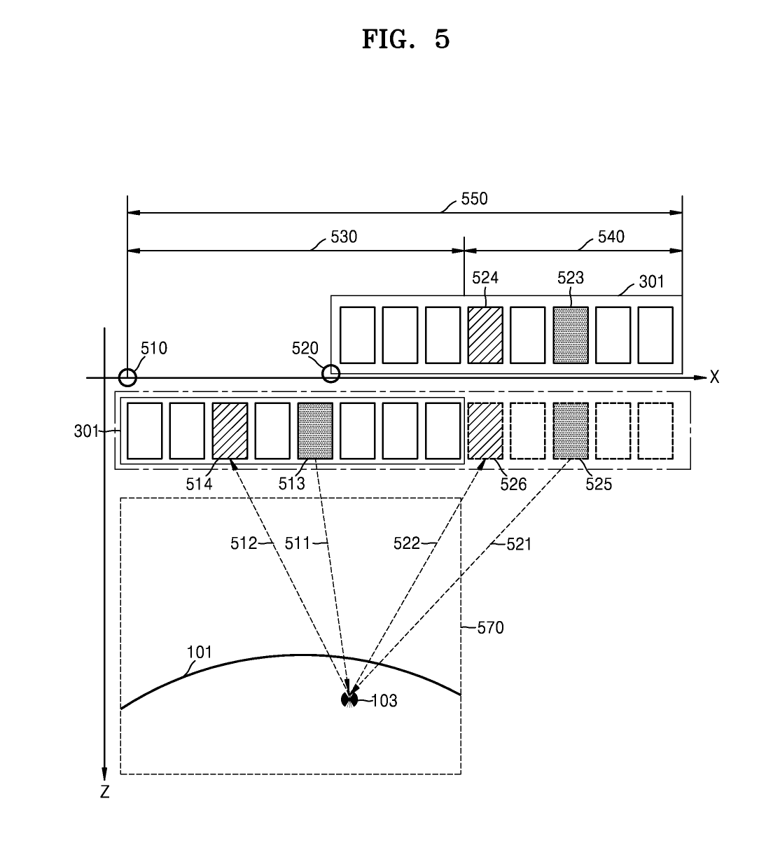

FIG. 5 is a diagram illustrating an ultrasound imaging operation using synthetic aperture focusing according to an exemplary embodiment. In detail, FIG. 5 illustrates an operation of generating an ultrasound image by synthetic aperture focusing of ultrasound signals transmitted and received by using the probe 301 located at different positions.

The ultrasound diagnostic apparatus 300 according to an exemplary embodiment may include the controller 305 that may perform control to acquire at least one piece of ultrasound data by synthetic-focusing at least one first ultrasound echo signal acquired by locating the probe 301 at the first position and at least one second ultrasound echo signal acquired by locating the probe 301 at the second position. The ultrasound diagnostic apparatus 300 according to an exemplary embodiment may include the controller 305 that may perform control such that the probe 301 is formed to have a synthetic aperture corresponding to the sum of an aperture 530 of the probe 301 at the first position and an aperture 540 of the probe 301 at the second position.

In detail, in FIG. 5, the position of a start point of the probe 301 changes from a first position 510 to a second position 520. An axis parallel to the arrangement direction of the transducers of the probe 301 is defined as an x axis, and a direction from the probe 301 toward the object 101 (i.e., a direction perpendicular to the x axis) is defined as a z axis; however, exemplary embodiments are not limited thereto. Also, in FIG. 5, the z axis represents a direction from the surface of the object 101 toward a center thereof. A reference point of the position of the probe 301 may be any point such as a left end, a right end, or a center of the probe 301 (hereinafter, it is assumed that the reference point of the position of the probe 301 is the left end of the probe 301).

While the probe 301 is located at the first position 510, a transducer 513 transmits an ultrasound signal. An ultrasound signal 511 transmitted at the first position 510 is reflected from an inside 103 of the object 101 and is received as an ultrasound echo signal 512 by a transducer 514 of the probe 301 located at the first position 510. The transducer 514 receiving the ultrasound echo signal might be not the only transducer receiving the ultrasound echo signal in response to the transducer 513 transmitting the ultrasound signal, and one or more other transducers may receive the ultrasound echo signal in response to the transducer 513 transmitting the ultrasound signal.

While the probe 301 is located at the second position 520 that is different from the first position 510, a transducer 523 transmits an ultrasound signal. An ultrasound signal 521 transmitted at the second position 520 is reflected from the inside 103 of the object 101 and is received as an ultrasound echo signal 522 by a transducer 524 of the probe 301 located at the second position 520. For convenience, FIG. 5 illustrates that the position of the probe 301 on the z axis at the first position 510 is different from the position of the probe 301 on the z axis at the second position 520. However, the position of the probe 301 on the z axis at the first position 510 may be identical to the position of the probe 301 on the z axis at the second position 520, and the probe 301 may be moved only on the x axis. In detail, the x-axis position of a transducer 525 transmitting an ultrasound signal at the second position 520 may correspond to the x-axis position of the transmission transducer 523, and the z-axis position of the transducer 525 may correspond to the z-axis position of the transducer 513 transmitting an ultrasound signal at the first position 510. Likewise, the x-axis position of a transducer 526 receiving an ultrasound echo signal at the second position 520 may correspond to the x-axis position of the reception transducer 524, and the z-axis position of the transducer 526 may correspond to the z-axis position of the transducer 514 receiving an ultrasound echo signal at the first position 510. The transducer 524 receiving the ultrasound echo signal might be not the only transducer receiving the ultrasound echo signal in response to the transducer 523 transmitting the ultrasound signal, and one or more other transducers may receive the ultrasound echo signal in response to the transducer 523 transmitting the ultrasound signal.

By using synthetic aperture focusing, the ultrasound transceiver 302 may generate ultrasound data of the inside 103 of the object 101 based on the ultrasound signal transmitted and received at the first position 510 and the ultrasound signal transmitted and received at the second position 520. For example, focusing may be performed based on the total distance that is equal to the sum of a distance from the transmission transducer to the inside 103 of the object 101 and a distance from the inside 103 of the object 101 to the reception transducer. A time taken from transmission to reception may be obtained by using Equation 1 below.

$${t}_{p} ( i , j ) = \frac{\vert \vec{{r}_{p}} - \vec{{r}_{e}} ( i ) \vert + \vert \vec{{r}_{p}} - \vec{{r}_{r}} ( j ) \vert}{v} \qquad Equation 1$$

On the x axis and the z axis of FIG. 5, when \vec{rₚ} is a vector value from a first point to the inside 103 of the object 101, \vec{rₑ}(i) is a vector value from a predetermined first point to the position of the ith transmission transducer, \vec{rᵣ}(j) is a vector value from a predetermined first point to the position of the jth reception transducer, and v is the speed of an ultrasound signal, |\vec{rₚ}−\vec{rₑ}(i)| may be a straight distance from the transmission transducer 513 at the first position 510 to the inside 103 of the object 101 and |\vec{rₚ}−\vec{rᵣ}(j)| may be a straight distance from the reception transducer 514 at the first position 510 to the inside 103 of the object 101. Thus, tₚ (i, j) may be a time taken for the ultrasound signal, which is transmitted by the ith transmission transducer 513, to be reflected from the inside of the object 101 and received by the jth reception transducer 514. Based on the time tₚ(i, j), the ultrasound data y_{f}(\vec{rₚ}) focused on the inside 103 of the object 101 located at the position \vec{rₚ} on a plane formed by the x axis and the z axis may be obtained by using Equation 2 below.

y_{f}(\vec{rₚ})=Σⱼ₌₁^{N}Σᵢ₌₁^{M}(a(tₚ(i,j),i,j)×yᵣ(tₚ(i,j),i,j) Equation 2

Herein, yᵣ(tₚ(i, j), i, j) denotes ultrasound data in each reception transducer when the jth reception transducer 514 receives, after the time tₚ(i, j), the ultrasound echo signal related to the ultrasound signal transmitted by the ith transmission transducer 513. Also, receive-focusing may be used by applying a proper time delay to the time tₚ(i, j). M denotes the number of times an ultrasonic wave is transmitted, and N denotes the number of transducers. a(tₚ(i, j), i, j) denotes a weight function (apodization) to be applied to the ultrasound data yᵣ(tₚ(i, j), i, j), which will be described later. However, synthetic aperture focusing used in the exemplary embodiments is not limited to such a method.

Thus, since the final synthetic-aperture-focused ultrasound data is generated by using the ultrasound data acquired by transmitting an ultrasound signal by the probe 301 at the first position 510 and transmitting an ultrasound signal by the probe 301 at the second position that is different from the first position, an original aperture size 530 of the probe 301 is expanded to an aperture size 550 that is increased by the distance from the first position 510 to the second position 520, as illustrated in FIG. 5. That is, the probe 301 with the aperture size 530 may provide the same effect as a probe with the aperture size 550 when acquiring an ultrasound image 570. As described above, since the resolution of an ultrasound image is proportional to the aperture size, the resolution of an ultrasound image may be increased by increasing the aperture size. Thus, the ultrasound diagnostic apparatus 300 according to an exemplary embodiment may increase the resolution of an ultrasound image by acquiring a synthetic aperture image through an aperture that is larger than the actual aperture of the probe 301.

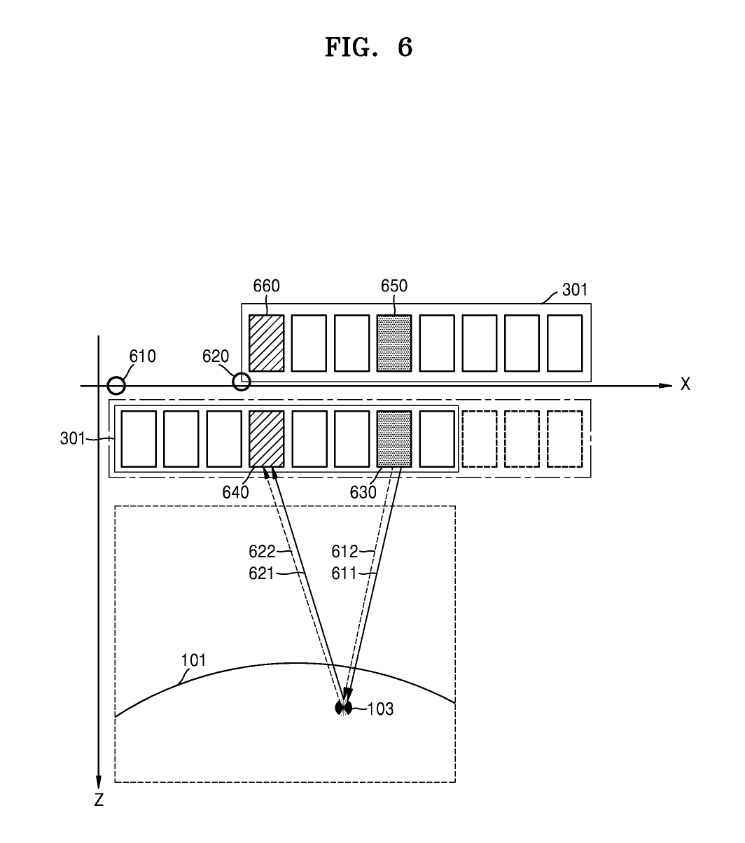

FIG. 6 is a diagram illustrating an ultrasound imaging operation using synthetic aperture focusing according to an exemplary embodiment. In detail, FIG. 6 illustrates a case where an ultrasound echo signal transmitted and received at a first position 610 or a second position 620 is determined to be the same, according to an exemplary embodiment. An ultrasound diagnostic apparatus according to an exemplary embodiment may include the image processor 303 that may delete one of information about a first ultrasound echo signal and information about a second ultrasound echo signal when at least one first ultrasound echo signal and at least one second ultrasound echo signal are determined to be the same. The ultrasound diagnostic apparatus according to an exemplary embodiment may include the image processor 303 that may apply a predetermined weight to an ultrasound echo signal, which is determined to be the same, among at least one of at least one first ultrasound echo signal and at least one of at least one second ultrasound echo signal.

When the probe 301 transmits an ultrasound signal 611 from a transducer 630 to the inside 103 of the object 101 at a first position 610, an ultrasound echo signal 621 reflected from the inside 103 of the object 101 may be received by a transducer 640. When the probe 301 is located at a second position 620 that is different from the first position 610 and transmits an ultrasound signal 612 from a transducer 650 to the inside 103 of the object 101 and a transducer 660 receives an ultrasound echo signal 622 related to the ultrasound signal 612, predetermined processing (e.g., leaving ultrasound data corresponding to only one ultrasound echo signal or applying a predetermined weight to relevant ultrasound data) may be performed on information about the ultrasound echo signal that is determined to have the same yᵣ(tₚ(i, j), i, j) value in Equation 2.

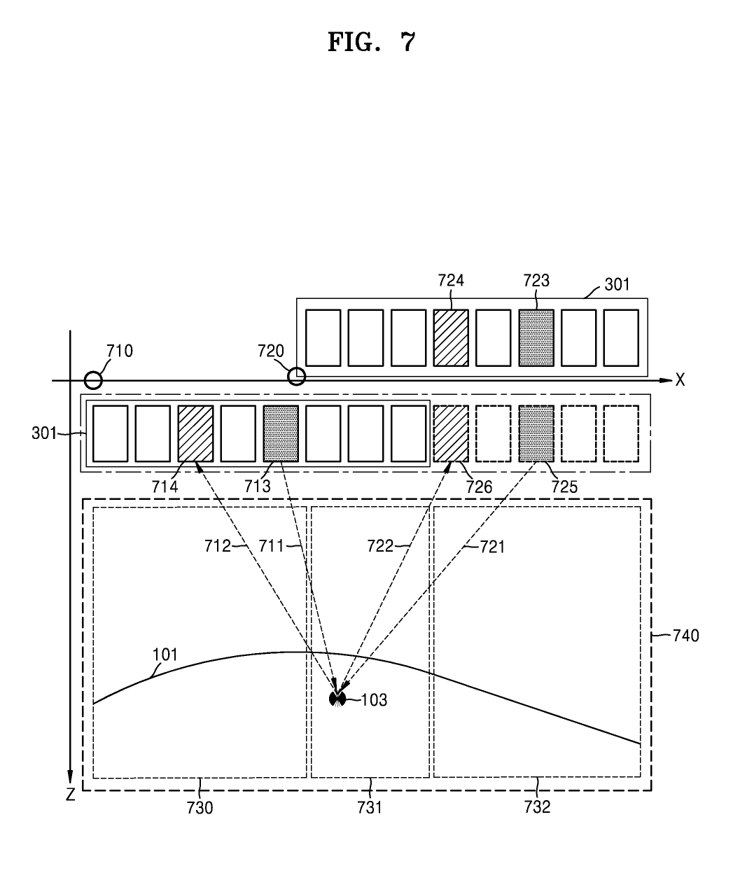

FIG. 7 illustrates generating a panoramic image by using ultrasound data generated at different positions via the movement of the probe 301, according to an exemplary embodiment.

In detail, while the probe 301 is located at a first position 710, a transducer 713 transmits an ultrasound signal. An ultrasound signal 711 transmitted at the first position 710 is reflected from the inside 103 of the object 101 and is received as an ultrasound echo signal 712 by a transducer 714 of the probe 301.

While the probe 301 is located at a second position 720 that is different from the first position 710, a transducer 723 transmits an ultrasound signal. An ultrasound signal 721 transmitted at the second position 720 is reflected from the inside 103 of the object 101 and is received as an ultrasound echo signal 722 by a transducer 724 of the probe 301. For convenience, FIG. 7 illustrates that the position of the probe 301 on the z axis at the first position 710 is different from the position of the probe 301 on the z axis at the second position 720. However, it is assumed that the position of the probe 301 on the z axis at the first position 710 is identical to the position of the probe 301 on the z axis at the second position 720. Thus, the x-axis position of a transducer 725 transmitting an ultrasound signal at the second position 720 may correspond to the position of the transmission transducer 723, and the z-axis position of the transducer 725 may correspond to the position of the transducer 713 transmitting an ultrasound signal at the first position 710. Likewise, the x-axis position of a transducer 726 receiving an ultrasound echo signal at the second position 720 may correspond to the x-axis position of the reception transducer 724, and the z-axis position of the transducer 726 may correspond to the z-axis position of the transducer 714 receiving an ultrasound echo signal at the first position 710.

As illustrated in FIG. 7, a panoramic ultrasound image 740 may be generated by connecting a previously-generated ultrasound image portion 730 and an ultrasound image portion 732 that is newly generated when the probe 301 moves from the first position 710 to the second position 720. Image processing on a portion 731 at which two ultrasound images 730 and 732 join or overlap may be performed by using a panorama imaging technology; however, exemplary embodiments are not limited thereto.

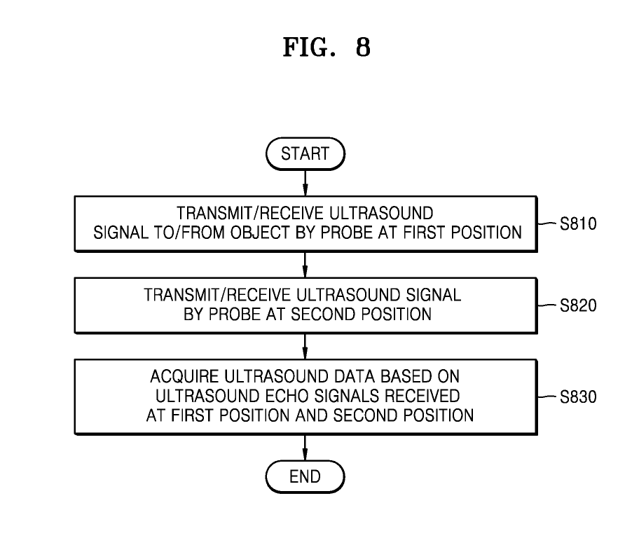

FIG. 8 is a flowchart of an ultrasound data acquiring method using synthetic aperture focusing to acquire ultrasound data by transmitting and receiving ultrasound signals at different positions via the movement of the probe 301, according to an exemplary embodiment.

The ultrasound data acquiring method according to an exemplary embodiment may include: transmitting and receiving at least one ultrasound signal to and from the object 101 by the probe 301 at a first position; transmitting and receiving at least one ultrasound signal to and from the object 101 by the probe 301 at a second position that is different from the first position; and acquiring at least one piece of ultrasound data based on at least one first ultrasound echo signal received at the first position and at least one second ultrasound echo signal received at the second position. Also, in the ultrasound data acquiring method according to an exemplary embodiment, the acquiring of the at least one piece of ultrasound data may include acquiring the at least one piece of ultrasound data by synthetic aperture focusing of the at least one first ultrasound echo signal and the at least one second ultrasound echo signal.

In detail, in operation S810, the probe 301 transmits and receives at least one ultrasound signal to and from the object at the first position.

In operation S820, the probe 301 moves to the second position that is different from the first position and transmits and receives at least one ultrasound signal to and from the object.

In operation S830, at least one piece of ultrasound data is acquired based on the first ultrasound echo signal acquired in operation S810 and the second ultrasound signal acquired in operation S820. The at least one piece of ultrasound data may be acquired by any synthetic aperture focusing. Without the need to transmit ultrasound signals from a plurality of transducers to one focus region by simultaneous focusing as illustrated in FIG. 2, the transducers in the probe 301 may transmit ultrasound signals sequentially as illustrated in FIG. 3. An ultrasound data acquiring method may be performed based on the ultrasound imaging operation described with reference to FIG. 5. Whether the position of the probe 301 is changed may be determined by attaching a position sensor to the ultrasound diagnostic apparatus; however, exemplary embodiments are not limited thereto.

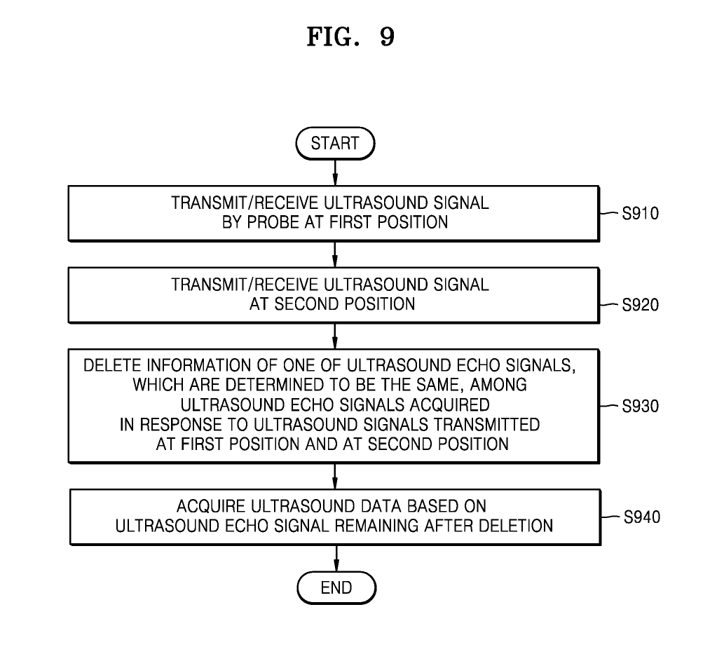

FIG. 9 is a diagram illustrating an ultrasound imaging operation using synthetic aperture focusing according to an exemplary embodiment. In detail, FIG. 9 is a flowchart of a method of acquiring ultrasound data based on information about an ultrasound echo signal other than an ultrasound echo signal that is determined to be the same, among at least one first ultrasound echo signal acquired in response to an ultrasound signal transmitted to an object at a first position and at least one second ultrasound echo signal acquired in response to an ultrasound signal transmitted to the object at a second position, according to an exemplary embodiment.

In detail, in operation S910, the probe 301 transmits and receives at least one ultrasound signal to and from the object at the first position.

In operation S920, the probe 301 moves to the second position that is different from the first position and transmits and receives at least one ultrasound signal to and from the object.

In operation S930, at least one first ultrasound echo signal acquired in operation S910 and at least one second ultrasound echo signal acquired in operation S920 are compared, and one of the ultrasound echo signals, which are determined to be the same, is deleted. For example, when the probe 301 moves from the first position to the second position and transmits and receives ultrasound signals by a plurality of transducers included in the probe 301, if the positions of the transducers transmitting ultrasound signals are identical to each other, acquired ultrasound echo signals may be determined to be the same. In this case, information of one of the first ultrasound echo signal and the second ultrasound echo signal, which are determined to be the same, may be deleted, and ultrasound data may be generated based on the information of the other of the first ultrasound echo signal and the second ultrasound echo signal (operation S940).

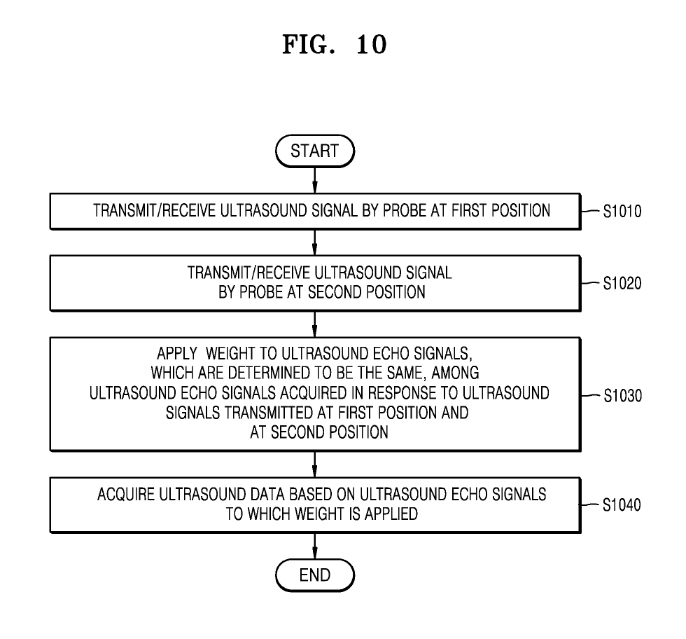

FIG. 10 is a flowchart of a method of acquiring ultrasound data by applying a predetermined weight to information about a first ultrasound echo signal and a second ultrasound echo signal determined to be the same, among at least one of at least one first ultrasound echo signal and at least one of at least one second ultrasound echo signal, according to an exemplary embodiment.

In detail, in the ultrasound diagnostic method according to an exemplary embodiment, the acquiring of the at least one piece of ultrasound data may include applying a predetermined weight to the ultrasound echo signal, which is determined to be the same, among at least one of the at least one first ultrasound echo signal and at least one of the at least one second ultrasound echo signal.

In operation S1010, the probe 301 transmits and receives at least one ultrasound signal to and from the object at the first position.

In operation S1020, the probe 301 moves to the second position that is different from the first position and transmits and receives at least one ultrasound signal to and from the object.

In operation S1030, at least one first ultrasound echo signal acquired in operation S1010 and at least one second ultrasound echo signal acquired in operation S1020 are compared, and one of the ultrasound echo signals, which are determined to be the same, is deleted. For example, when the probe 301 moves from the first position to the second position and transmits and receives ultrasound signals by a plurality of transducers included in the probe 301, if the positions of the transducers transmitting and receiving ultrasound signals are identical to each other, some of acquired ultrasound echo signals may be determined to be the same. In this case, a predetermined weight may be applied to information about the first ultrasound echo signal or information about the second ultrasound echo signal, which are determined to be the same as each other, and ultrasound data may be generated based on the ultrasound echo signals to which the predetermined weight have been applied (operation S1040).

According to an exemplary embodiment, an apodization value may be used to apply a weight to ultrasound signal information. Referring to FIG. 5, a(tₚ(i, j), i, j) in Equation 2 denotes an apodization value as a weight function to be applied to each yᵣ(tₚ(i, j), i, j) that is ultrasound signal information in each reception transducer, when the jth reception transducer 514 receives the ultrasound echo signal related to the ultrasound signal transmitted by the ith transmission transducer 513 after the time tₚ(i, j) that is taken for the ultrasound signal, which is transmitted by the ith transmission transducer 513, to be reflected from the inside 103 of the object 101 and received by the jth reception transducer 514. For example, when information about the first ultrasound echo signal and information about the second ultrasound echo signal are determined to be the same, a weight of 50% is applied to each of the information about the first ultrasound echo signal and the information about the second ultrasound echo signal. Thus, the sum of the information about the first ultrasound echo signal and the information about the second ultrasound echo signal may be used as information about the ultrasound echo signal corresponding to 100% of information about the ultrasound echo signal that is not determined to be the same as the information about the first ultrasound echo signal or the second ultrasound echo signal. Also, by applying a proper time delay to the time tₚ(i, j) that is a factor determining the apodization value, a weight may be applied in a receive focusing process. The apodization method described as a method of applying a weight is merely an example, and exemplary embodiments are not limited thereto.

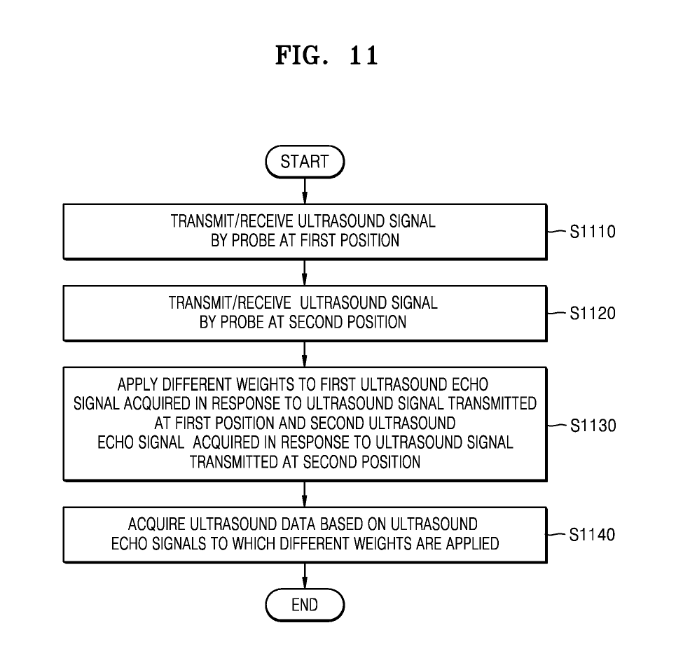

FIG. 11 is a flowchart of a method of acquiring ultrasound data by applying different weights to a first ultrasound echo signal and a second ultrasound echo signal, according to an exemplary embodiment.

In detail, in the ultrasound diagnostic method according to an exemplary embodiment, the acquiring of the at least one piece of ultrasound data may include applying different weights to the at least one first ultrasound echo signal and the at least one second ultrasound echo signal.

In operation S1110, the probe 301 transmits and receives at least one ultrasound signal to and from the object at the first position.

In operation S1120, the probe 301 moves to the second position that is different from the first position and transmits and receives at least one ultrasound signal to and from the object.

In operation S1130, ultrasound data is generated by applying different weights to the at least one first ultrasound echo signal acquired in operation S1010 and the at least one second ultrasound echo signal acquired in operation S1020. Unlike the case of FIG. 10, whether information about the first ultrasound echo signal and information about the second ultrasound echo signal are the same is not determined. It is determined whether the information about the first ultrasound echo signal is received at predetermined intervals. For example, the ultrasound echo signal corresponding to the ultrasound signal transmitted at the first position may be determined to be the first ultrasound echo signal, and the ultrasound echo signal related to the ultrasound signal transmitted at the nth time after transmission of the ultrasound signal may be again determined to be the first ultrasound echo signal.

In operation S1140, ultrasound data is acquired based on the at least one first ultrasound echo signal and the at least one second ultrasound echo signal to which the different weights have been applied in operation S1130.

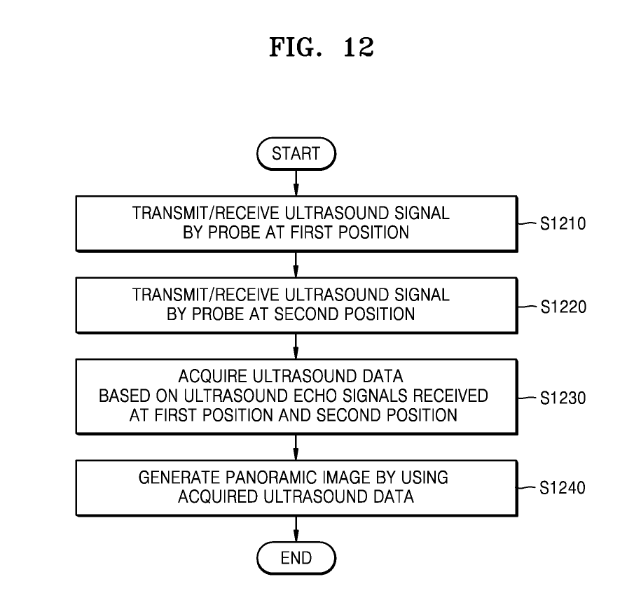

FIG. 12 is a flowchart of a method of generating a panoramic image by using ultrasound data acquired by synthetic aperture focusing of ultrasound signals transmitted and received by using the probe 301 located at different positions, according to an exemplary embodiment. In detail, in the ultrasound diagnostic method according to an exemplary embodiment, the acquiring of the at least one piece of ultrasound data may include generating a panoramic image including the ultrasound data generated based on the at least one first ultrasound echo signal and the at least one second ultrasound echo signal.

In operation S1210, the probe 301 transmits and receives at least one ultrasound signal to and from the object at the first position.

In operation S1220, the probe 301 moves to the second position that is different from the first position and transmits and receives at least one ultrasound signal to and from the object.

In operation S1230, at least one piece of ultrasound data is acquired based on the at least one first ultrasound echo signal acquired in operation S1210 and the at least one second ultrasound echo signal acquired in operation S1220. The at least one piece of ultrasound data may be acquired by any synthetic aperture focusing. Without the need to transmit ultrasound signals from a plurality of transducers to one focus region by simultaneous focusing as illustrated in FIG. 2, the transducers in the probe 301 may transmit ultrasound signals sequentially as illustrated in FIG. 3. An ultrasound data acquiring method may be performed based on the ultrasound imaging operation described with reference to FIG. 5. Whether the position of the probe 301 is changed may be determined by attaching a position sensor to the ultrasound diagnostic apparatus; however, exemplary embodiments are not limited thereto.

In operation S1240, a panoramic image is generated based on the at least one piece ultrasound data acquired in operation S1230. A panoramic image acquiring method may be performed as described with reference to FIG. 7.

As described above, according to one or more of exemplary embodiments, it is possible to provide an ultrasound image that enables the user to diagnose an object by using an improved image resolution.

The foregoing exemplary embodiments and advantages are merely exemplary and are not limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An ultrasound diagnostic apparatus comprising:
an ultrasound transceiver configured to supply a driving signal to at least one transducer included in a probe, and acquire ultrasound data corresponding to a first ultrasound echo signal and a second ultrasound echo signal received by the at least one transducer, in response to transmitting a first ultrasound signal and a second ultrasound signal to an object, respectively;
a controller configured to perform control to acquire the ultrasound data based on the first ultrasound echo signal acquired in response to transmitting the first ultrasound signal at a first position of the probe and the second ultrasound echo signal acquired in response to transmitting the second ultrasound signal at a second position of the probe, the second position being different from the first position; and
an image processor configured to generate an ultrasound image by using the ultrasound data.

2. The ultrasound diagnostic apparatus of claim 1, wherein the controller is configured to perform the control to acquire the ultrasound data by synthetic aperture focusing of the first ultrasound echo signal and the second ultrasound echo signal.

3. The ultrasound diagnostic apparatus of claim 1, wherein the controller is configured to perform the control such that the probe is formed to have a synthetic aperture corresponding to a sum of an aperture of the probe at the first position and an aperture of the probe at the second position.

4. The ultrasound diagnostic apparatus of claim 1, wherein the image processor is configured to delete information of one of the first ultrasound echo signal and the second ultrasound echo signal, in response to determining the first ultrasound echo signal being the same as the second ultrasound echo signal.

5. The ultrasound diagnostic apparatus of claim 1, wherein the image processor is configured to apply a weight to the first ultrasound echo signal and the second ultrasound echo signal, in response to determining the first ultrasound echo signal being the same as the second ultrasound echo signal.

6. The ultrasound diagnostic apparatus of claim 1, wherein the image processor is configured to generate the ultrasound data by applying different weights to the first ultrasound echo signal and the second ultrasound echo signal.

7. The ultrasound diagnostic apparatus of claim 1, wherein the image processor is configured to generate a panoramic image including the ultrasound data generated based on the first ultrasound echo signal and the second ultrasound echo signal.

8. An ultrasound diagnostic method comprising:
transmitting a first ultrasound signal to an object by a probe at a first position;
transmitting a second ultrasound signal to the object by the probe at a second position that is different from the first position; and
acquiring ultrasound data based on a first ultrasound echo signal received in response to the transmitting the first ultrasound signal at the first position and a second ultrasound echo signal received in response to the transmitting the second ultrasound signal at the second position.

9. The ultrasound diagnostic method of claim 8, wherein the acquiring the ultrasound data comprises:
acquiring the ultrasound data by synthetic aperture focusing of the first ultrasound echo signal and the second ultrasound echo signal.

10. The ultrasound diagnostic method of claim 8, wherein the acquiring of the ultrasound data comprises:
performing a calculation to acquire ultrasound data by using the probe formed to have a synthetic aperture corresponding to a synthesis of an aperture of the probe at the first position and an aperture of the probe at the second position.

11. The ultrasound diagnostic method of claim 8, wherein the acquiring the ultrasound data comprises:
deleting information of one of the first ultrasound echo signal and the second ultrasound echo signal, in response to determining the first ultrasound echo signal being the same as the second ultrasound echo signal.

12. The ultrasound diagnostic method of claim 8, wherein the acquiring of the ultrasound data comprises:
applying a weight to the first and second ultrasound echo signals, in response to determining the first ultrasound echo signal being the same as the second ultrasound echo signal.

13. The ultrasound diagnostic method of claim 8, wherein the acquiring the ultrasound data comprises:
acquiring the ultrasound data by applying different weights to the first ultrasound echo signal and the second ultrasound echo signal.

14. The ultrasound diagnostic method of claim 8, wherein the acquiring the ultrasound data comprises:
generating a panoramic image including the ultrasound data generated based on the first ultrasound echo signal and the second ultrasound echo signal.

15. A non-transitory computer-readable recording medium that stores a program which, when executed by a computer, causes the computer to perform the ultrasound diagnostic method of claim 8.
